(19) United States
(12) Patent Application Publication
(10) Pub. No.: US 2013/0315801 A1
(43) Pub. Date: Nov. 28, 2013

(54) CARTRIDGE FOR A BLOOD TREATMENT APPARATUS

(71) Applicants: Lennart Jonsson, Bjarred (SE); Ragnar Tryggvason, Loddekopinge (SE)

(72) Inventors: Lennart Jonsson, Bjarred (SE); Ragnar Tryggvason, Loddekopinge (SE)

(73) Assignee: Gambro Lundia AB, Lund (SE)

(21) Appl. No.: 13/991,612

(22) PCT Filed: Nov. 11, 2011
(86) PCT No.: PCT/EP2011/069926

§ 371 Date: Aug. 2, 2013

Related U.S. Application Data
(60) Provisional application No. 61/420,341, filed on Dec. 7, 2010.

(30) Foreign Application Priority Data
Dec. 7, 2010 (SE) 1051286-1

(51) Int. Cl. B01L 3/00

(52) U.S. Cl. CPC A61M 1/367
USPC 422/554

(57) ABSTRACT

A cartridge configured to be connected to a blood treatment apparatus, the cartridge including a cartridge inlet, a cartridge outlet and chambers each of which is configured to hold a concentrate in powder form. A flow controller is configured to provide a flow passage from the cartridge inlet to a single selected chamber of the chambers and from the single selected chamber to the cartridge outlet. The flow controller is movable between a plurality of positions, such that a position of the flow controller defines which of the plurality of chambers is the single selected chamber.

### TECHNICAL FIELD

The invention relates to a cartridge that is configured to be connected to a blood treatment apparatus. The cartridge comprises a cartridge inlet, a cartridge outlet and a plurality of chambers of which each may hold a concentrate in powder form.

### BACKGROUND ART

Today blood treatment apparatuses are used for extracorporeal blood treatment which involves drawing blood from a patient, treating the blood and returning the treated blood to the patient. For this purpose an extracorporeal blood flow circuit (blood circuit) is used which is connected to a blood vessel access of the patient, typically via one or more access devices such as needles or catheters inserted into an arm of the patient. Depending on the method of blood treatment, the blood may be withdrawn from the patient, passed through the blood side of a blood treatment unit (dialyzer) and returned to the patient via the same or another blood vessel access device. In hemodialysis, as one example of an extracorporeal blood treatment, simultaneously a dialysis fluid circuit (fluid circuit) draws a treatment fluid (dialysis fluid) from a fluid source, passes the treatment fluid through the blood treatment unit where the blood is treated, and disposes used treatment fluid in a drain.

Other examples of extracorporeal blood treatment includes hemodiafiltration and hemofiltration, and various techniques are used for these different types of blood treatment. A common way to prepare the treatment fluid in connection with hemodialysis and hemodiafiltration operations, or the replacement fluids in connection with hemodiafiltration and hemofiltration operations, is to mix a fluid concentrate or a concentrate in powder form with pure water.

In some cases the concentrate to be mixed with water is prepared in centralized preparation plants and is thereafter transferred to the point of treatment in large kegs or other containers. However, to ensure that the concentrate remains stable and/or bacteria-free, techniques are employed where the concentrate is continuously prepared during a session of blood treatment of a patient.

The continuous preparation typically includes feeding a stream of water into a container that holds a concentrate in powder form that is dissolved by the water. The substance typically has the form of a powder. The water and the dissolved substance form a fluid concentrate, and various valves are then used for extracting the fluid concentrate from the container and continuously mixing the fluid concentrate with additional water, such that a desired composition of a treatment fluid is formed. Typically, more than one concentrate have to be diluted with water and mixed together to achieve a treatment fluid with all required components.

Containers may also be used for batch-wise preparation of a treatment fluid. In this kind of preparation all components that are needed for a treatment fluid have to be put in the container before the water is added. Thus, the concentration of the different components in relation to each other cannot be changed once the container has been closed by the manufacturer. This is in contrast to the continuous preparation, where each component has its own container with fluid concentrate or concentrate in powder form to be dissolved in water. The output from each container is a fluid concentrate, and a number of such concentrates are to be mixed and diluted to achieve a treatment fluid with all required components.

Examples of techniques that use containers can be found in patent documents U.S. Pat. No. 4,784,495 and U.S. Pat. No. 6,444,174, of which U.S. Pat. No. 6,444,174 shows a container for batch-wise preparation.

Known techniques are generally capable of preparing a fluid concentrate for the treatment fluid. However, it is believed that present techniques may be improved in the sense that a supply of a fluid concentrate should be facilitated for different blood treatment scenarios, while still allowing use of a standard-type of container that efficiently holds a concentrate in powder form for forming the fluid concentrate.

### SUMMARY

It is an object of the invention to at least partly overcome one or more limitations of the prior art. In particular, it is an object to provide a standard-type of container that may hold a concentrate in powder form, while still supporting different scenarios of blood treatment.

Hence a cartridge is provided, which is configured to be connected to a blood treatment apparatus and which comprises a cartridge inlet, a cartridge outlet and a plurality of chambers where each chamber is configured to hold a concentrate in powder form. The cartridge comprises a flow controller that is configured to provide a flow passage from the cartridge inlet, into a single selected chamber of the plurality of chambers and from the single selected chamber to the cartridge outlet. The flow controller is movable between a plurality of positions, such that a position of the flow controller defines which of the plurality of chambers is the selected chamber.

The cartridge is advantageous in that the total amount of substance taken from the cartridge may be adapted to the requirements of a certain blood treatment scenario. This may be accomplished by the flow controller, by allowing it to move such that chamber after chamber is used as the selected chamber during a blood treatment session. The number of chambers used should correspond to the number of chambers that in combination hold an amount of a concentrate in powder form that is required for the certain blood treatment session. Thus, different blood treatment scenarios are efficiently supported in that relatively accurate amounts of substance may be used.

Another advantage with the cartridge is that substance in any chamber(s) of the plurality of chambers that has not been the selected chamber may be used at a later time during another session of blood treatment. This may be accomplished since the unselected chamber has not provided a flow passage, i.e. its substance is dry, which ensures that it remains stable and/or bacteria-free.

The flow controller may be rotatable in relation to the plurality of chambers. Also, a rotational position of the flow controller may define which of the plurality of chambers is the selected chamber.

In one embodiment the flow controller comprises a cut-out that is arranged to face the selected chamber, for providing the flow passage from the selected chamber to the cartridge outlet.

Each of the plurality of chambers may comprise a respective opening, such that the cut-out can face an opening of the selected chamber, for providing the flow passage from the selected chamber to the cartridge outlet.

The flow controller may comprise an elongated member that extends from an upper section of the cartridge to a lower section of the cartridge. In one embodiment a lower end section of the elongated member comprises the cut-out.

Each of the plurality of chambers may comprise a respective fluid opening for allowing the flow controller to define which of the plurality of chambers is the selected chamber.

The cartridge may comprise a lid that is rotatable in relation to the plurality of chambers. In one embodiment the lid is connected to the flow controller, such that a position of the lid defines the position of the flow controller. Additionally, in one embodiment the lid may comprise the cartridge inlet, and a position of the lid may then define the flow passage from the cartridge inlet into the selected chamber.

The flow controller may comprise the cartridge inlet, and a position of the flow controller may define the flow passage from the cartridge inlet into the selected chamber.

The cartridge inlet and the cartridge outlet may be configured to be held by a respective cartridge support of a blood treatment apparatus.

Each of the plurality of chambers may contain sodium chloride or sodium bicarbonate.

According to another aspect a blood treatment apparatus is provided, which comprises a cartridge that implements any of the above described features. The blood treatment apparatus comprises a first cartridge support that is configured to hold the cartridge inlet, and a second cartridge support that is configured to hold the cartridge outlet.

Still other objectives, features, aspects and advantages of the invention will appear from the following detailed description, from the attached claims as well as from the drawings.

### BRIEF DESCRIPTION OF THE DRAWINGS

Embodiments of the invention will now be described, by way of example, with reference to the accompanying schematic drawings, in which

FIG. 1 illustrates a blood treatment apparatus with a cartridge for holding a concentrate in powder form,

FIG. 2a is a cross-sectional view of a cartridge support of the blood treatment apparatus of FIG. 1,

FIG. 2b is a perspective view of a stand-alone unit for a cartridge,

FIG. 3 is an exploded view of the cartridge of FIG. 1,

FIG. 4 is perspective view of the cartridge of FIG. 1,

FIG. 5 is an exploded, partial view of the cartridge of FIG. 1,

FIG. 6 is an enlarged, partial view of the cartridge of FIG. 1,

FIG. 7 is a cross-sectional view of another embodiment of a cartridge, and

FIG. 8 is a cross-sectional, partial view of still another embodiment of the cartridge.

### DETAILED DESCRIPTION

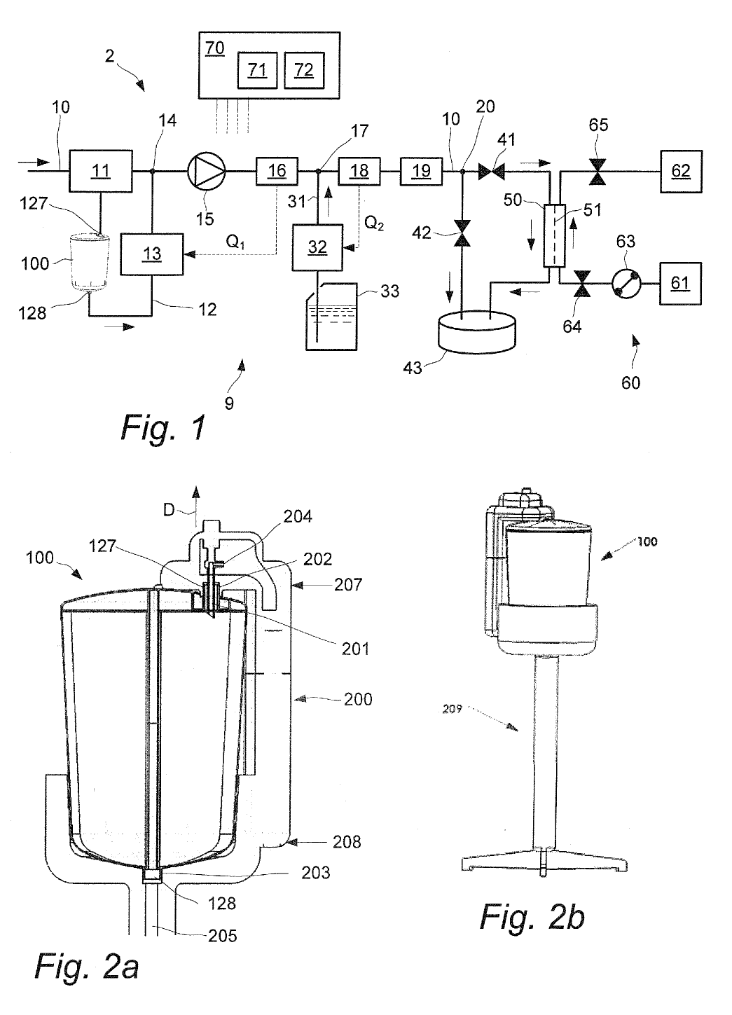

With reference to FIG. 1 an embodiment of a blood treatment apparatus 2 for extracorporeal blood treatment, such as dialysis, is illustrated. The blood treatment apparatus 2 (dialysis machine) comprises a blood treatment unit 50 and a blood circuit 60 with a blood pump 63 arranged to draw blood from a blood source 61, pass the blood through the blood treatment unit 50 which then may treat the blood and deliver the treated blood to a target vessel 62.

The blood circuit 60 is divided into an upstream blood circuit that connects the blood source 61 to the blood treatment unit 50, while the downstream blood circuit connects the blood treatment unit 50 with the target vessel 62. A first blood clamp 64 is arranged upstream the blood treatment unit 50 and a second blood clamp 65 is arranged downstream the blood treatment unit 50. The blood clamps 64, 65 may open and close the blood circuit 60 as controlled by a processing unit 70 of the blood treatment apparatus 2, such that a flow from the blood source 61, through the blood treatment unit 50 and to the target vessel 62 may be controlled. Generally, the blood pump 63 may be controlled by the processing unit 70 for enabling a desired flow of blood.

For reasons of clarity, signal paths between the processing unit 70 and various components it controls have been omitted from the drawings.

Within the blood treatment unit 50 a semi-permeable membrane 51 is present and separates the blood treatment unit 50 into a dialysis fluid compartment through which treatment fluid may be passed, and a blood compartment through which blood may be passed. When treatment fluid and blood are passed through the blood treatment unit 50 the membrane 51 allows the treatment fluid to interact with the blood in a manner known within the art.

The configuration of the blood circuit 60 and the blood treatment unit 50 may include various other components and control units generally present in blood treatment apparatuses. The blood source 61 and target vessel 62 may be a patient that receives blood treatment, but may also be bags of blood that are handled by operators. Even though the blood source 61 and the target vessel 62 are shown as separate units, they may be one and the same unit. The blood treatment apparatus 2 may be arranged to operate so as to perform single-needle dialysis and/or double-needle dialysis, and may therefore include additional components conventionally used for this purpose.

The blood treatment unit 50 and the blood circuit 60 may be arranged as a common, disposable unit in the form of a unitary device that may be disconnected from the blood treatment apparatus 2 and discarded once a session of blood treatment of a patient is completed. When a new patient shall undertake treatment by the blood treatment apparatus 2, a new and similar common unit is connected to the apparatus 2 and a new treatment session may commence.

The blood treatment apparatus 2 comprises a fluid circuit 9 for passing the treatment fluid through the blood treatment unit 50. The treatment fluid is continuously prepared during a blood treatment session, which is done by the fluid circuit 9 that in this embodiment prepares the treatment fluid from one concentrate in powder form and one concentrate in liquid form. As is known, it is possible replace the concentrate in liquid form with another concentrate in powder form and one concentrate in liquid form. The two concentrates in powder form may then be dissolved by using similar techniques.

The fluid circuit 9 comprises a main fluid conduit 10. The main fluid conduit 10 has, in a downstream direction, a liquid reservoir 11, a first mixing point 14, a fluid pump 15, a first measuring device 16, a second mixing point 17, a second measuring device 18, a pH meter 19, a separation point 20 and a fluid valve 41. After the fluid valve 41 the main fluid conduit 10 is connected to the blood treatment unit 50, and treatment fluid that has passed the blood treatment unit 50 is conveyed to a drain 43 via a fluid circuit downstream the blood treatment unit 50.

A first concentrate conduit 12 is connected between the liquid reservoir 11 and the first mixing point 14. The first concentrate conduit 12 comprises, in a direction from the liquid reservoir 11 to the first mixing point 14, a cartridge 100 and a regulating device 13. The regulating device 13 may have the form of a conventional pump.

The cartridge 100 has a cartridge inlet 127 and a cartridge outlet 128 and holds a concentrate in powder form. In detail, for the illustrated embodiment the cartridge 100 comprises one single inlet in form of the cartridge inlet 127 and one single outlet in form of the cartridge outlet 128.

The concentrate in powder form held by the cartridge 100 comprises dry concentrates of NaCl (sodium chloride) or NaHCO₃ (sodium bicarbonate) in powder form. The substance comprises all dry forms such as e.g. powder, granulate and particulate form, regardless of grain size. However, the grain size is in most cases larger than 100 micrometer, and preferably in the range 130-500 micrometer. In this context, the term “powder” refers to all particles with sizes ranging from 50 micrometer to 800 micrometer.

As an alternative the cartridge may hold glucose with the same grain size as mentioned above.

In operation, a portion of the water in the liquid reservoir 11 is drawn off through the first concentrate conduit 12 and is introduced into the cartridge inlet 127 which is arranged at an upper section of the cartridge 100. The introduced water is conducted downwardly toward a bottom of the cartridge 100 where the cartridge outlet 128 is arranged. When the water is conducted in the cartridge 100, a substantially saturated solution of the powder concentrate in water is obtained, to thus produce a first concentrate fluid (dissolved substance) which is then conducted from the cartridge outlet 128 and introduced into the main fluid conduit 10 at the first mixing point 14. The regulating device 13 that is arranged intermediate the cartridge 100 and the first mixing point 14 controls a flow of the first concentrate fluid from the cartridge 100 to the main fluid conduit 10 where the first concentrate fluid is mixed with water from the liquid reservoir 11.

The fluid pump 15 conveys the fluid in the main fluid conduit 10 and may include functionality for deaerating the fluid as well as for properly mixing the first concentrate fluid from the first concentrate conduit 12 with the purified water from the liquid reservoir 11.

The first measuring device 16, which may have the form of a conductivity meter, monitors the composition of the fluid in the main fluid conduit 10 and controls the regulating device 13 in the first concentrate conduit 12. In this manner, it is possible to accurately control the ultimate mixture of the first fluid concentrate from the first concentrate conduit 12 with purified water that is conducted from the liquid reservoir 11 to the first mixing point 14. This is accomplished even if the concentrate in powder form were to dissolve to different extents or degrees of saturation in the cartridge 100.

Instead of performing conductivity measurements, the first measuring device 16 could measure a different property or parameter, such as temperature, pH, or even some other parameter. Typically, the first measuring device 16 controls the regulating device 13 in the first concentrate conduit 12 by sending a proper control signal Q₁ to the regulating device 13.

At the second mixing point 17 a second concentrate fluid is introduced into the main fluid conduit 10 via a second concentrate conduit 31. The sec- and concentrate conduit 31 comprises a regulating device 32 capable of drawing the second concentrate fluid from a concentrate source 33. The second concentrate fluid is in this example in liquid form and may comprise any substances that is not present in the cartridge but needed for a patient that undertakes blood treatment.

The regulating device 32 in the second concentrate conduit 31 may be a conventional pump or any other suitable device that may feed a fluid into the second mixing point 17. The flow of the second concentrate fluid through the second concentrate conduit 31 is regulated with the aid of the second measuring device 18, for example by measuring a conductivity and using a control signal Q₂ for controlling the regulating device 32.

For ultimate monitoring of the prepared solution, a pH meter 19 may be installed in the main fluid conduit 10. If conductivity measured by e.g. the measuring devices 16, 18, or a pH, temperature, or any other parameter utilized for controlling the flow of concentrates through the main fluid conduit 10 do not correspond with desired values, the prepared fluid is passed via a bypass valve 42 directly to the drain 43. This is done until the regulators 13, 32 are adjusted such that desired values are obtained.

When, on the other hand, all of the parameters are correct or in accordance with their desired values, the prepared solution is passed as treatment fluid via the fluid valve 41 and through the blood treatment unit 50. As mentioned, after the treatment fluid is passed through the blood treatment unit 50 it is conveyed to the drain 43.

Thus, in this embodiment, two concentrates are conducted to the main fluid conduit 10 at two separate mixing points 14, 17 in the main fluid conduit 10 for mixing with fluid conducted therein. Additional conductivity meters or other control devices may be included in the main fluid conduit 10, in the first concentrate conduit 12 and/or in the second concentrate conduit 31 to ensure accurate monitoring of the composition of the prepared solution.

Additionally, the fluid circuit 9 as well as the blood circuit 60 may implement known techniques and standards, and may thus include various other components and control units generally used in blood treatment apparatuses, such as filters, flow meters, pressure sensors, additional pumps and clamps etc. The various components and functions of the blood treatment apparatus 2 may be initiated and controlled by the processing unit 70

For this purpose the processing unit 70 typically includes one or more processor(s) such that a central processing unit 71 which may execute software instructions, i.e. computer program code that carry out relevant functions and operations of the blood treatment apparatus 2, either alone or in cooperation with other components. For this purpose the processing unit 70 may include a computer-readable memory 72 that stores the software instructions. These may for development convenience be written in a high-level programming language such as Java, C, and/or C++ but also in other programming languages, such as, but not limited to, interpreted languages.

With reference to FIG. 2a, the blood treatment apparatus 2 comprises a cartridge holder 200 that supports the cartridge 100. The cartridge holder 200 has a first cartridge support 202 and a second cartridge support 203. The first cartridge support 202 is connectable to the cartridge inlet 127 and the second cartridge support 203 is connectable to the cartridge outlet 128. FIG. 2a shows the cartridge 100 in place, supported in the cartridge holder 200 and connected to the first cartridge support 202 and to the second cartridge support 203. The cartridge supports 202, 203 hold the cartridge 100 in position in the first concentrate conduit 12. The cartridge holder 200 may via an inlet 204 receive the purified water from the liquid reservoir 11 and may via an outlet 205 feed the first concentrate fluid to the first mixing point 14, via the regulating device 13 in the first concentrate conduit 12.

An upper section 207 of the cartridge holder 200 may be moved in a direction D from a lower section 208 of the cartridge holder 200, which removes the first support 202 from the cartridge inlet 127 such that the complete cartridge 100 may be removed from the cartridge holder 200. A new cartridge may thereafter be placed in the cartridge holder 200 and the upper section 207 of the cartridge holder 200 may then be moved in a direction opposite the direction D towards the lower section 208 of the cartridge holder 200, for fixing the new cartridge in the cartridge holder 200.

With reference to FIG. 2b, it is possible to support the cartridge 100 by using a stand-alone cartridge holder 209. The stand-alone cartridge holder 209 may comprise the same components as the cartridge holder of FIG. 2a and may perform the same operations. However, the stand-alone unit 209 is not a part of the blood treatment apparatus. Instead fluid conduits are used for connecting the cartridge 100 to the liquid reservoir 11 and to the regulating device 13.

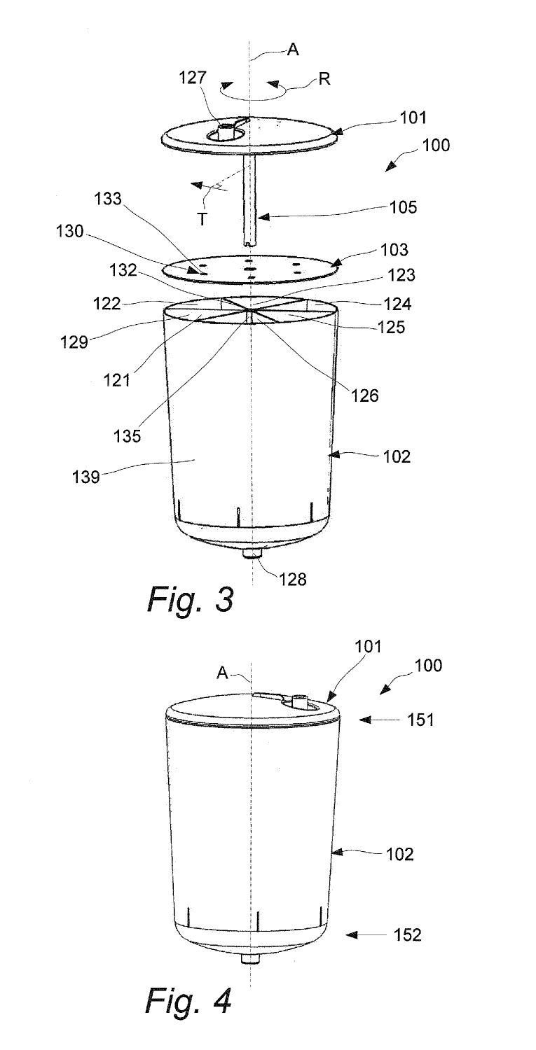

With further reference to FIGS. 3 and 4 the cartridge 100 is illustrated in further detail. The cartridge 100 comprises a body 102 with a first chamber 121, a second chamber 122, a third chamber 123, a fourth chamber 124, a fifth chamber 125 and a sixth chamber 126. Thus, the cartridge 100 comprises a plurality of chambers which in this example is illustrated by the six chambers 121-126. However, the plurality of chambers may comprise another number of chambers such as 2 to 10 chambers or even more. Specifically, the plurality of chambers may comprise at least five chambers. Each of the chambers 121-126 is configured to hold a concentrate in powder form for preparing the first concentrate fluid previously described.

The body 102 of the cartridge has the shape of a truncated cone with a diameter that is slightly larger at an upper section 151 of the body 102 in comparison with a lower section 152 of the body 102. Apart from the cartridge outlet 128 the lower section 152 of the body 102 is closed, while the upper section 151 of the body 102 is open.

The body 102 is symmetrical about a geometrical centre axis A and comprises a cylindrical channel 135 at its center. The channel 135 extends from the upper section 151 to the cartridge outlet 128 at the lower section 152, and defines an elongated through hole 132 which accordingly extends from the upper section 151 to the cartridge outlet 128. Six symmetrically arranged chamber walls extend (in a radial direction) from the channel 135 to a wall 139 that forms the outer boundaries of the body 102, and extend (in a longitudinal direction) from the upper section 151 to a bottom of the body 102.

A first chamber wall of the six chamber walls is indicated by reference numeral 129, and separates the first chamber 121 from the second chamber 122. The chambers 121-126 are defined by the chambers walls, and the chambers 121-126 have equal volumes since the chamber walls are symmetrically arranged in the body 102. However, it is possible to arrange the chamber walls asymmetrically such that the chambers have different volumes.

A circular plate 103 covers the body 102 such that the chambers 121-126 are closed at the upper section 151. However, six holes, or inlet passages, are symmetrically arranged in the plate 103 at a position above a respective chamber. Each hole is sealed by a membrane. For example, the plate 103 has its first hole 130 arranged above the first chamber 121, and this first hole 130 is covered by a membrane 133. The other holes are similar with the first hole apart from being arranged over a respectively different chamber.

Thus, each of the plurality of chambers 121-126 may be associated with a respective inlet passage. The inlet passages may in turn be covered by at least one membrane that is configured to be e.g. punched for providing the flow passage from the cartridge inlet 127 into a selected chamber, as will be described below.

As an alternative to cover each of the six holes with a respective membrane, one membrane that covers the full plate 103 may be used.

The plate 103 is covered by a lid 101 that protects the plate 103. The lid 101 is also circular and comprises the cartridge inlet 127. As may be seen from FIG. 3, the uppermost part of the body 102 (closest to the plate 103) has a diameter that corresponds to a diameter of the lid 101.

The lid 101 is rotatable in relation to the body 102, which means that the lid 101 is rotatable in relation to the chambers 121-126 as well. This also means that the lid 101 is rotatable in relation to the plate 103, as the plate 103 is fixed in relation to the chambers 121-126. In detail, the lid 101 is rotatable in a direction R (see FIG. 3) about the geometrical axis A. For providing the rotation between the lid 101 and the body 102, the lid 101 may be attached to the body 102 by a snap-fitting that provides a small play such the lid 101 and the body 102 are rotatable in relation to each other.

The plate 103 is however fixed to the body 102. Moreover, the cartridge inlet 127 is arranged at a radial distance from the geometrical axis A.

This radial distance is equal to a radial distance by which each of the holes in the plate 103 is arranged from the same axis A. Thus, by properly rotating the lid 101 the cartridge inlet 127 may be arranged above any of the holes in the plate 103. Preferably, each of the holes in the plate 103 are centered above the respective chamber.

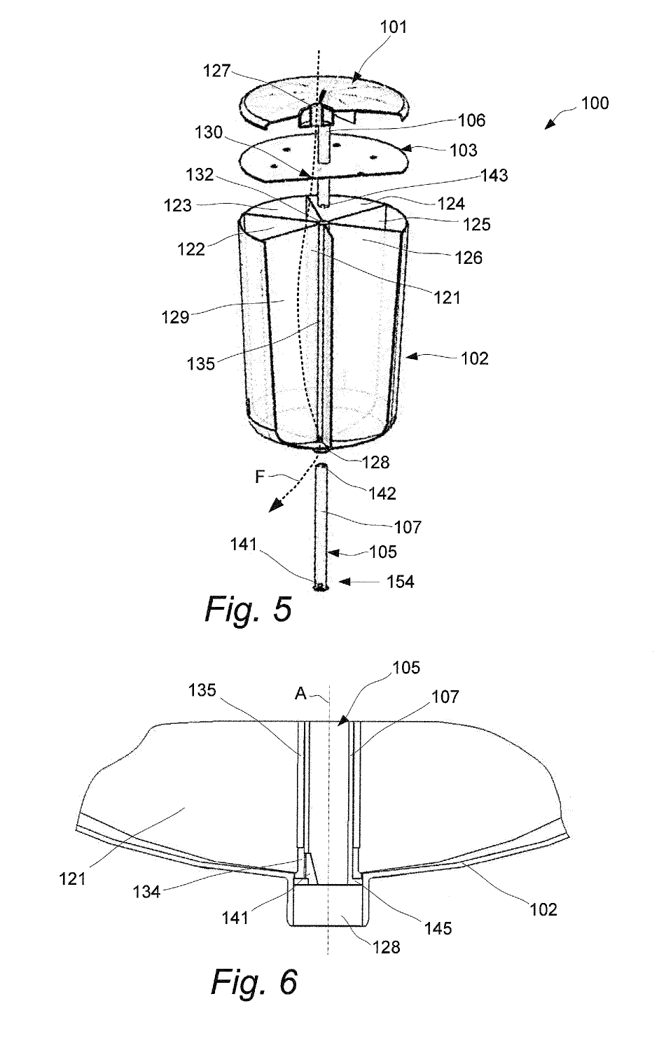

With reference to FIG. 5 the cartridge 100 comprises a flow controller 105 that provides a flow passage F from the cartridge inlet 127, into a selected chamber 121 of the chambers 121-126 via the opening 130 above the selected chamber 121, and from the selected chamber 121 to the cartridge outlet 128. In this embodiment the first chamber 121 is illustrated as the selected chamber.

The flow controller 105 is movable between a plurality of positions, such that a position of the flow controller 105 defines which of the plurality of chambers 121-126 is the selected chamber 121. From this follows that the number of positions the flow controller 105 may move between corresponds to the number of chambers in the cartridge 100.

The flow controller 105 comprises an elongated member which in turn comprises a first elongated member 106 and a second elongated member 107 that is coupled to the first elongated member 106. By virtue of the coupling between the elongated members 106, 107 a rotational movement of the first elongated member 106 may be transferred to the second elongated member 107. In combination the elongated members 106, 107 extend from the upper section 151 of the cartridge 100 to the lower section 152 of the cartridge 100. Specifically, the flow controller 105 in form of the elongated members 106, 107 extend from the lid 101 to the cartridge outlet 128.

It is possible to integrate the elongated members 106, 107 into one unit. However, it appears that two shorter elongated member may provide some advantages in terms of manufacturing in comparison with one relatively longer elongated member.

The coupling between the first elongated member 106 and the second elongated member 107 may be accomplished by arranging a small groove 143 at an end of the first elongated member 106 that shall be coupled to the second elongated member 107, while a small protrusion 142 that fits into the groove 143 is arranged at an end of the second elongated member 107 that shall be coupled to the first elongated member 106. Of course, other means for coupling of the elongated members 106, 107 to each other may be used with equal result.

The flow controller 105, or more specifically the first elongated member 106, is fixedly attached to the lid 101, and since the lid 101 is rotatable in relation to the chambers 121-126, the flow controller 105 in form of the members 106, 107 is rotatable in relation to the chambers 121-126 as well. This means that the flow controller 105 is rotatable in the direction R (see FIG. 3) about the geometrical axis A.

The flow controller 105 in form of the elongated members 106, 107 has a circular, cylindrical shape and is arranged within the through hole 132, such that the flow controller 105 extends from the lid 101 to the cartridge outlet 128. The elongated members 106, 107 are in this embodiment hollow, which means that the flow controller 105 may have the shape of a pipe that is connected to the lid 101, is inserted in the channel 135 at the center of the cartridge 100, and extends from the lid 101 to the cartridge outlet 128. The elongated members 106, 107 may also be solid or only the lower part of the sec- and elongated member 107 may be hollow.

With reference to FIG. 6 a partial, close-up view of lower parts of the cartridge 100 and flow controller 105 are illustrated. As may be seen from the figure, the flow controller 105 extends to the cartridge outlet 128 at the very bottom of the cartridge 100. The second elongated member 107 that is part of the flow controller 105 has a small flange 145 that provides an efficient seal against the body 102, and has a cut-out in form of a recess 141 that is arranged to face the selected chamber (here exemplified by the first chamber 121). The recess 141 is implemented for providing the flow passage F from the selected chamber 121 to the cartridge outlet 128.

Each of the chambers 121-126 comprises a respective fluid opening, such as fluid opening 134 for the first chamber 121. The openings are similarly arranged at a lowest section of the respective chambers. The openings are provided for letting out the first concentrate fluid when the flow controller 105 has defined which of the plurality of chambers 121-126 is the selected chamber.

In detail, the selected chamber is the chamber that the recess 141 faces. When the recess 141 faces the selected chamber, which is exemplified by the first chamber 121, the recess 141 also faces the opening 134 of the selected chamber 121. When this occurs a flow path is provided from the selected chamber 121, out of the opening 134 of the selected chamber 121 and into the flow controller 105 (in form of the second elongated member 107) via the recess 141. When the flow controller 105 or its lower part has the form of a pipe, fluid may then pass from the flow controller 105 to the cartridge outlet 128. If the lower part of the flow controller 105 is solid, the recess 141 may be in the form of an obliquely cut away part to allow the fluid to pass to the cartridge outlet 128. It is to be noted that at the same time as the recess 141 faces the opening 134 of the selected chamber 121 and provides the fluid passage F to the cartridge outlet 128, the lower part of the second elongated member 107 also seals the openings from the rest of the chambers 122-126. Regardless of if the lower part of the second elongated member 107 is hollow or solid, the openings from the rest of the chambers 122-126 not being the selected chamber is sealed off from the cartridge outlet 128.

To prevent not dissolved particles from escaping from the cartridge a filter may be provided at the cartridge outlet 128 or preferably in connection with the openings from the chambers. This filter may be in the form of a slit filter.

The cartridge inlet 127 is aligned with the recess 141, which means that both the cartridge inlet 127 and the recess 141 face that same chamber when the lid 101 is rotated from one chamber to the other. Hence, the selected chamber is the chamber that both the cartridge inlet 127 and the recess 141 face.

In preparation of a blood treatment session the cartridge inlet 127 is positioned at the selected chamber 121, i.e. the lid 101 is rotated until the cartridge inlet 127 is centered above the hole 130 in the plate 103 that is centered above the selected chamber 121. In the embodiment shown in FIG. 2a, the first cartridge support 202 that is connected to the cartridge inlet 127 is in such a position that it in this case actually is the body 102 that is rotated and the lid 101 with the cartridge inlet 127 that is in a fixed position. Thereafter a puncher 201 of the cartridge holder 200 (see FIG. 2a) is pushed into the cartridge inlet 127 and through the membrane 133 of the hole 130 which thereby provides the flow passage from the cartridge inlet 127 into the selected chamber 121 via the hole 130. The hole 130 may here be referred to as an inlet passage.

Since the lid 101 is connected to the flow controller 105 the position of the lid 101 also defines the position of the flow controller 105. Since the inlet 127 and the recess 141 are aligned, the recess 141 automatically faces the opening 134 of the selected chamber 121 when the cartridge inlet 127 is centered at the hole 130 associated with the selected chamber 121. As previously explained, the holes, such as hole 130, are centered above its associated chamber, and the plate 103 that defines the holes is fixed to the body 102 such that the plate can not rotate with the lid 101.

The full flow passage F from the cartridge inlet 127, via the selected chamber 121 to the cartridge outlet 128 is shown in FIG. 5.

During a blood treatment session the flow controller 105 (and the lid 101) is moved in relation to the chambers 121-126 (or the chambers 121-126 are moved in relation to the flow controller 105 and the lid 101) such that one of the chambers becomes the selected chamber. The membrane associated with the selected chamber is thereafter punched by the puncher 201, purified water is fed into the selected chamber via the puncher 201, and the first concentrate fluid is supplied to the first mixing point 14 as described above. Instead of using the puncher 201 for feeding purified water into the selected chamber, another water-feeding fluid conduit may be used. In this case the puncher 201 should be removed when the membrane has been punched.

When it is decided that the selected chamber is empty or soon will be empty, the puncher 201 alternatively the water-feeding fluid conduit is extracted from the cartridge inlet 127 and the flow controller 105 is moved to a new position such that next chamber is the selected chamber. The membrane of the new selected chamber is then punched and purified water is fed into the new selected chamber such that the first concentrate fluid can be supplied to the first mixing point 14.

Since the flow controller 105 may move between positions that make one chamber after the other “the selected chamber”, the cartridge 100 and/or the flow controller 105 is configured to provide sequential use of the chambers 121-126. Thus, the chambers 121-126 are configured to be used sequentially during one or more blood treatment operations.

The process of moving the flow controller such that a next chamber is the selected chamber goes on until the blood treatment session is complete. Content of any chamber that has not been the selected chamber may be used at another blood treatment session. The process of moving the flow controller can be manual but can also be automated, as exemplified below.

If the content of all chambers in the cartridge are used before a treatment session is complete, the flow of fluid may be temporarily stopped while the cartridge is replaced with a new one.

In this context, it should be noted that the flow controller 105 being movable in relation to the fluid chambers 121-126 is the same as the fluid chambers 121-126 being movable in relation to the flow controller 105. Also, the flow controller 105 may be seen as integral with the cartridge 100, since it forms a unitary device with the cartridge.

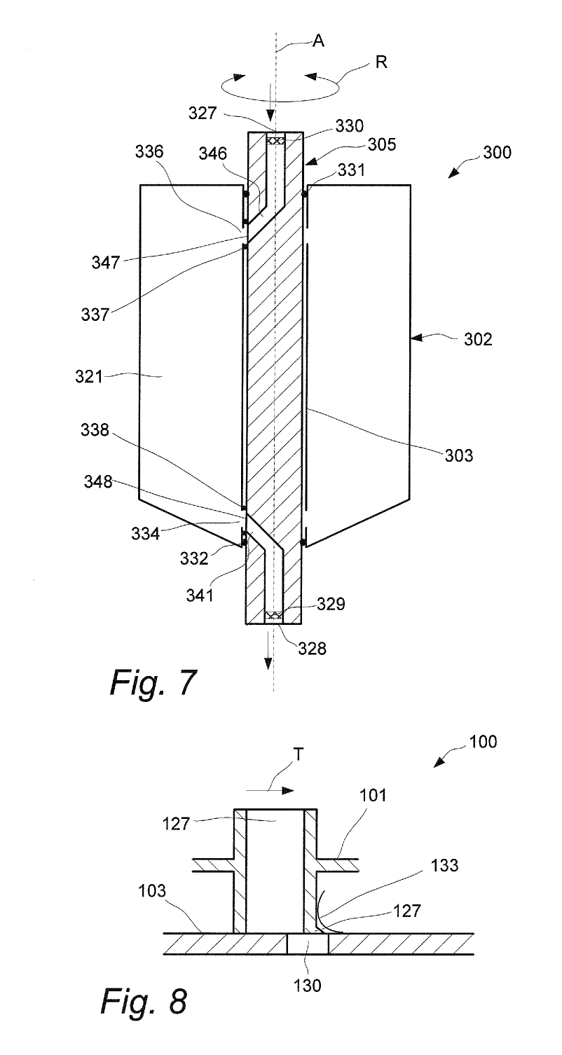

With reference to FIG. 7 an alternative embodiment of a cartridge 300 is described. In this embodiment the cartridge 300 comprises a body 302 with six chambers just like the cartridge described in connection with the previous figures. A position of a flow controller 305 defines which of the chambers is the selected chamber.

In the illustrated embodiment the selected chamber is indicated by reference numeral 321, and each chamber has an upper chamber inlet and a lower chamber outlet, such as chamber inlet 336 and chamber outlet 334 of the selected chamber 321. The flow controller 305 is elongated, extends through a through hole 303 in the body 302 and is rotatable in relation to the body 302 in a direction R about a geometrical axis A. This means that the flow controller 305 is rotatable in relation to the chambers of the cartridge 300. Each of the through hole 303 of the body 302 and the flow controller 305 has a circular cross section.

The flow controller 305 is fixed to the body 302 in a longitudinal direction (parallel with the axis A) by a first bearing 331 and a second bearing 332. The bearings can also act as seal rings.

At a first end, which can be seen as an upper end, the flow controller 305 comprises a cartridge inlet 327 in form of a hole that extends into the flow controller 305 in a direction along the axis. A cut-out in form of a passage 346 is arranged between the inlet 327 and a side of the flow controller 305, such that a flow of fluid may pass into the flow controller 305 via the cartridge inlet 327 and out from the flow controller 305 at an opening 347 in the side of the flow controller 305. The passage 346 is arranged such that the opening 347 faces the chamber inlet 336, which allows a fluid to pass from the flow controller 305 and into the selected chamber 321. A gasket 337 may be fixed to the flow controller 305 and arranged around the opening 347 for providing a liquid tight seal between the flow controller 305 and the wall around the chamber inlet 336 body 302.

A filter 330, such as a slit filter, may be arranged in the inlet 327 or at a suitable location in the passage 346, or alternatively at the chamber inlet 336. The filter 330 prevents a content in powder form from escaping from the chambers, but allows a liquid to pass through.

At a second end, which can be seen as a lower end, the flow controller 305 comprises a cartridge outlet 328 in form of a hole that extends into the flow controller 305 in a direction along the axis A. A cut-out in form of a passage 341 is arranged between the side of the flow controller 305 and the outlet 328, such that a flow of fluid may pass from the selected chamber 321, into an opening 348 defined by the low passage 341, through the passage 341 and to the cartridge outlet 328 where the fluid can leave the cartridge 300. This means that the passage 341 is arranged such that the opening 348 faces the chamber outlet 334 of the selected chamber 321. A gasket 338 may be fixed to the flow controller 305 and arranged around the opening 348 for providing a liquid tight seal between the flow controller 305 and the wall around the chamber outlet 334. The outlet 328 may comprise a filter 329 that is similar with the filter 330 at the inlet 327, i.e. it lets liquid pass through the outlet 328 but prevents powder from passing. Such a filter 329, may be a slit filter, and may alternatively be arranged at a suitable location in the passage 341, or alternatively at the chamber outlet 334.

During operation, purified water is conveyed into the cartridge inlet 327, further down through the passage 346 and into the selected chamber 321 via the opening 347 and the chamber inlet 336. The water dissolves a content of the selected chamber 321. The liquid formed leaves the selected chamber 321 via the chamber outlet 334, passes through the opening 348, passes through the passage 341 and exits the cartridge 300 via the cartridge outlet 328.

Obviously, a fluid passage is provided only when the openings 347, 348 face an inlet and outlet of a chamber, and since the openings may only face an inlet/outlet of one chamber, fluid may then not pass into any chamber but the selected chamber. By rotating the flow controller 305 a next chamber becomes the “selected” chamber.

From above follows that e.g. the passage 341 has the same function as the recess 141 of FIG. 6. Thus, the passage 341 may bee seen as a “recess”.

With reference to FIG. 8 another embodiment of the cartridge is shown where an alternative to the puncher described above is used. FIG. 8, which is best viewed in combination with FIG. 3, illustrates an enlarged, cross sectional view of the cartridge inlet 127, the lid 101 and the plate 103. In this embodiment each inlet passage of the chambers is provided with a membrane, such as membrane 133 at the inlet passage 130. When the lid 101 is rotated for choosing another chamber as the selected chamber, the cartridge inlet 127 moves in a tangential direction T (see FIG. 3 and FIG. 8). During this movement an extension 127 of the lid abuts against the plate 103 and tears off the membrane 133 that covers the inlet passage 130. In this case the membrane 133 may be attached to the plate 103 by an adhesive. Naturally, in this case another water-feeding fluid conduit may replace the puncher for feeding purified water into the selected chamber.

As an alternative to tearing off the membrane a cutting device may be arranged on the lid for opening the membrane by a cutting movement.

As indicated, an alternative or complement to using the membrane(s) one or more slit filters may be used. For example, a conventional slit filter (not shown) may replace the membrane 133 at the inlet passage 130. The slit filter then prevents any powder from passing through the inlet passage while liquid may pass. A slit filter may also be arranged at the cartridge inlet 127 and/or cartridge outlet 128, which is mentioned for the embodiment described in connection with FIG. 7.

The cartridge may also comprise caps (not shown) that cover the cartridge inlet and cartridge outlet during transportation and storage.

In one embodiment of the blood treatment apparatus a mechanism (not shown) is provided for rotating the body 102 of the cartridge 100 in relation to the lid 101, which is equivalent with moving the flow controller 105 in relation to the chambers 121-126. The mechanism may comprise a set of rollers that abut to the body 102 such that the body is rotated when the rollers are rotated. The lid 101 should then be fixed to the cartridge holder 200 and the rollers may be controlled by the processing unit 70 for enabling an automatic selection of the chambers.

When automating the selection of chambers, a measurement device may be arranged in the first concentrate conduit 12. When this measurement device indicates that the first concentrate exhibits to low concentration levels, selection of a new chamber may be initiated. Additionally, the puncher 201 may be automated and controlled by the processing unit 70 for providing a fully automated system.

Typically, the cartridge including all its components may be made of a plastic material such as a suitable polyolefin like polyethylene or polypropylene. Other suitable materials may be polycarbonates or modifications of e.g. polyethylene terephthalate (PET), for example PET modified by copolymerization (PETG).

The volumes of the chamber of the cartridge may vary. It is also possible to adapt volumes of the chambers such that the chambers hold a certain weight of concentrate in powder form. For example, each of the chambers may have a volume that allows them to hold an amount of 600 g NaCl in powder form, i.e. the cartridge may hold in total 3600 g NaCl when six chambers are used. Since 1200 g NaCl is typically needed during a blood treatment session, the cartridge may then last for three treatment sessions before it must be replaced. A larger or smaller cartridge is of course achievable, for example a cartridge that may hold 4800-6000 g or even 8400-9600 g NaCl.

Suitable volumes for the chambers in case the cartridge holds NaHCO₃ may be volumes that allow each chamber to hold 325-360 g NaHCO₃, which means that the cartridge may hold 1950-2160 g NaHCO₃ in total when it has six chambers. Since 650-720 g NaHCO₃ is typically needed during a blood treatment session, the cartridge may then last for three treatment session before it must be replaced. A larger or smaller cartridge is of course achievable, such as a cartridge that may hold 2600-3600 g or even 4550-5760 g NaHCO₃.

Of course, the principles described herein for defining which of a plurality of chambers is a selected chamber may be employed in connection with other types of blood treatment apparatuses than the one described herein. Additionally, the cartridge is not restricted to use in connection with blood treatment since it may be employed for other types of medical applications that utilize containers for holding a medical substance.

Also, other techniques for providing the selection of a chamber may be used. Thus, although various embodiments of the invention have been described and shown, the invention is not restricted thereto, but may also be embodied in other ways within the scope of the subject-matter defined in the following claims.

1. A cartridge configured to be connected to a blood treatment apparatus, the cartridge comprising:
a cartridge inlet,
a cartridge outlet;
a plurality of chambers where each chamber is configured to hold a concentrate in powder form, and
a flow controller configured to provide a flow passage from the cartridge inlet into a single selected chamber of the plurality of chambers and from the single selected chamber to the cartridge outlet, wherein the flow controller is movable between a plurality of positions, such that a position of the flow controller defines which of the plurality of chambers is the selected chamber.

2. A cartridge according to claim 1, wherein the flow controller is rotatable in relation to the plurality of chambers.

3. A cartridge according to claim 1, wherein a rotational position of the flow controller defines which of the plurality of chambers is the selected chamber.

4. A cartridge according to claim 1, wherein the flow controller comprises a cut-out that is arranged to face the selected chamber, for providing the flow passage from the selected chamber to the cartridge outlet.

5. A cartridge according to claim 4, wherein each of the plurality of chambers comprises a respective opening, such that the cut-out may face an opening of the selected chamber, for providing the flow passage from the selected chamber to the cartridge outlet.

6. A cartridge according to claim 1, wherein the flow controller comprises an elongated member that extends from an upper section of the cartridge to a lower section of the cartridge.

7. A cartridge according to claim 4, wherein a lower end section of the elongated member comprises the cut-out.

8. A cartridge according to claim 1, wherein each of the plurality of chambers comprises a respective opening for allowing the flow controller to define which of the plurality of chambers is the selected chamber.

9. A cartridge according to claim 1, comprising a lid that is rotatable in relation to the plurality of chambers.

10. A cartridge according to claim 9, wherein the lid is connected to the flow controller, such that a position of the lid defines the position of the flow controller.

11. A cartridge according to claim 10, wherein the lid comprises the cartridge inlet, and a position of the lid defines the flow passage from the cartridge inlet into the selected chamber.

12. A cartridge according to claim 1, wherein the flow controller comprises the cartridge inlet, and a position of the flow controller defines the flow passage from the cartridge inlet into the selected chamber.

13. A cartridge according to claim 1, wherein the cartridge inlet and the cartridge outlet are configured to be held by a respective cartridge support of a blood treatment apparatus.

14. A cartridge according to claim 1, wherein each of the plurality of chambers contains sodium chloride or sodium bicarbonate.

15. A blood treatment apparatus comprising a cartridge according to claim 1, comprising a first cartridge support configured to hold the cartridge inlet and a second cartridge support configured to hold the cartridge outlet.

16. A cartridge for a blood treatment apparatus comprising:
a cartridge inlet configured to be in fluid communication with a source of fluid for the blood treatment apparatus;
a cartridge outlet configured to be in fluid communication with a blood treatment unit;
chambers each configured to hold a concentrate in powder form, and
a flow controller configured to establish a flow passage through a selected one of the chambers and both the cartridge inlet and the cartridge outlet, wherein a relative movement between the flow controller and at least one of the cartridge inlet and the cartridge outlet selects one of the chambers as the selected chamber.

17. The cartridge according to claim 16, wherein the movement of the flow controller is a relative rotational movement between the flow controller and the cartridge.

18. The cartridge according to claim 16, wherein the flow controller is manually moveable.

19. The cartridge according to claim 16 further comprising a cartridge body housing the chambers and wherein:
the flow controller includes a cartridge lid mounted to the cartridge body,
the cartridge inlet or cartridge outlet is mounted to the lid and is axially offset from an axis of the lid, and
relative rotation between the lid and the cartridge body aligns at least one of the cartridge inlet and the cartridge outlet with the selected chamber.

20. The cartridge of claim 16 further comprising:
a cartridge body housing the chambers;
a lid capping the chambers, wherein the cartridge inlet or cartridge outlet is fixed to the lid, and
a plate between the chambers and the lid, wherein the lid includes an opening corresponding to each of the chambers and relative movement between the plate and lid aligns one of the cartridge inlet and cartridge outlet with the opening in the plate of the selected chamber.

21. The cartridge according to claim 20, wherein each of the chambers includes an opening positioned to be align with the aperture in the plate while the chamber is the selected chamber.

22. The cartridge according to claim 16, wherein the flow controller comprises an elongated member between the chambers and extending longitudinally along the chambers, wherein the elongated member includes an upper section passage establishing fluid communication from the cartridge inlet to the selected chamber and a lower section passage establishing fluid communication from the selected chamber to the cartridge outlet.

23. The cartridge of claim 16 further comprising a flow blockage device blocking fluid flow through all of the chambers except for the selected chamber.

24. The cartridge of claim 16 further comprising a cartridge surface configured to abut at least one roller and a rotatable mount, wherein the cartridge rotates about the mount when driven by the at least one roller.

25. A method to provide a mixture of at least one concentrate and a liquid to a blood treatment unit in a blood treatment apparatus the method comprising:
storing the at least one concentrate in chambers of a cartridge;
connecting an inlet to the cartridge to a source of a liquid;
connecting an outlet from the cartridge to the blood treatment apparatus;
establishing a flow of the liquid through the inlet, a first chamber of the chambers, the outlet and to the blood treatment unit, wherein the at least one concentrate in the first chamber mixes with the liquid flowing to the blood treatment unit;
treating blood in a first period with the blood treatment apparatus, wherein the treatment includes passing the blood through the blood treatment unit while the liquid flows from the first chamber to the blood treatment apparatus;
after the first period, selecting a second chamber of the chambers by causing relative movement between the flow controller and at least one of the inlet and outlet for the cartridge, and
treating blood in a second period with the blood treatment apparatus, wherein the treatment includes passing the blood through the blood treatment unit while the liquid flows from the second chamber to the blood treatment apparatus.

26. The method of claim 25 wherein the selection of the second chamber is performed using a flow controller mounted in a cartridge housing for the chambers and the step of selecting the second chamber includes causing relative movement between the flow controller and the cartridge housing.

27. The method of claim 26, wherein the flow controller is mounted in a housing for the cartridge, and the relative movement is a rotational movement about an axis of the housing.

28. The method of claim 26 wherein the relative movement is performed manually.

29. The method of claim 26 wherein the relative movement is performed automatically.

30. The method of claim 25 further comprising monitoring a concentrate level in the liquid flowing to the blood treatment unit and performing the selection of the second chamber in response to detecting a concentrate level below a threshold level.

31. The method of claim 25 further comprising isolating the chambers other than the first chamber from the flow of the liquid during the first period, and isolating the chambers other than the second chamber from the flow of the liquid during the second period.

32. The method of claim 25 wherein the at least one concentrate stored in each of the chambers all have a similar composition.
